(19) United States
(12) Patent Application Publication
(10) Pub. No.: US 2006/0253127 A1
(43) Pub. Date: Nov. 9, 2006

(54) Endoluminal linear suturing device

(75) Inventor: David B. Bjerken, Marietta, GA (US)

Correspondence Address: MCKENNA LONG & ALDRIDGE LLP, 1900 K STREET, NW, WASHINGTON, DC, 20006, US

(73) Assignee: Bernard Medical, LLC, Marietta, GA (US)

(21) Appl. No.: 11/267,321

(22) Filed: Nov. 7, 2005

Related U.S. Application Data
(60) Provisional application No. 60/677,355, filed on May 4, 2005.
(60) Provisional application No. 60/698,941, filed on Jul. 14, 2005.
(60) Provisional application No. 60/697,544, filed on Jul. 11, 2005.
(60) Provisional application No. 60/677,345, filed on May 4, 2005.

(51) Int. Cl. A61B 17/10

(52) U.S. Cl. 606/139

(57) ABSTRACT

A device for suturing tissue of a patient includes a tube defining a bore extending longitudinally within the tube and at least one suction opening in the exterior surface. A path is arranged within an interior of the tube. A needle is arranged at least partially within the bore and is attachable to a suture. The suction openings are adapted to be connected to a vacuum source such that, when a vacuum is applied, tissue is drawn into the at least one suction openings and at least partially into the bore. The needle is adapted to advance through the tissue and along the path to place the suture through the tissue.

### CROSS-REFERENCE TO RELATED APPLICATIONS

This application claims priority to Provisional Patent Application No. 60,677,345, filed May 4, 2005; Provisional Patent Application No. 60/677,355, filed May 11, 2005; Provisional Patent Application No. 60/697,544, filed Jul. 11, 2005; and Provisional Patent Application No. 60/698,941, filed Jul. 14, 2005. The disclosures of each of the above-mentioned applications are hereby incorporated by reference.

### BACKGROUND OF THE INVENTION

1. Field of the Invention

The present invention relates generally to suturing devices and methods for using the suturing device to place suture or tissue fastening material within tissue, for example, within a body organ.

2. Discussion of the Related Art

Various types of surgical procedures are currently performed to investigate, diagnose, and treat diseases and conditions within patients, including conditions and diseases within the gastrointestinal system and within the heart and great vessels within the thorax. Procedures include, for example, the placement of sutures involved with the treatment of many kinds of conditions and diseases. A suture is any fastening material. Conventionally the placement of sutures involves an invasive surgery to access the suture areas. Such procedures are time consuming, involve difficult placements of suture, and subject the patient to trauma and prolonged recovery.

U.S. Pat. Nos. 6,464,707 and 6,558,400 are hereby incorporated by reference in their entirety.

### SUMMARY OF THE INVENTION

The present invention includes a suturing device for suturing within a subject, and includes an enclosure defining at least one suction port for receiving tissue within the enclosure to be sutured. A suture needle is adapted to follow a helical path within the bore of the tube thereby penetrating the drawn in tissue and incorporating the suture within the tissue. The tube of the device is adapted to dismantle longitudinally allowing the withdrawal of the device after the suture line has been placed.

### BRIEF DESCRIPTION OF THE DRAWINGS

FIGS. 1A-B illustrate a view of an exemplary embodiment of the present invention.

FIGS. 2A-B illustrate the exemplary embodiment shown in FIGS. 1A and 1B with one example of a suture needle used in accordance with the present invention.

FIGS. 3A-B illustrate one example of the components that make up the exemplary embodiment shown in FIGS. 1A and 1B.

FIGS. 4A-B illustrate another exemplary embodiment in accordance with the present invention.

FIGS. 5A-B illustrate yet another exemplary embodiment of the present invention.

FIG. 6 illustrates another exemplary embodiment of the present invention

FIGS. 7A-B illustrate two examples of suture needles used in accordance with the present invention.

FIGS. 8A-D illustrate further examples of suture needles used in accordance with the present invention.

FIGS. 9A-B illustrate one embodiment the removable partition shown in FIGS. 9A and B incorporated within the device of the present invention.

FIGS. 10A-B illustrate a cross-sectional view of one example of a removable partition placed within the tube's bore used in accordance with the present invention.

FIG. 11 illustrates two views of an embodiment of a pushrod used in accordance with the present invention.

FIG. 12 illustrates an embodiment of a ratchet gun used in accordance with the present invention.

FIG. 13 illustrates an embodiment of the present invention that utilizes inflatable balloons.

FIG. 14 illustrates another embodiment of the present invention that utilizes an inflatable balloon.

FIGS. 15A-B illustrate a method of practicing one embodiment of the present invention.

FIGS. 16A-B illustrate another example of a method of practicing one embodiment of the present invention.

FIG. 17 illustrates a running suture line resulting from a method of practicing one embodiment of the present invention.

FIGS. 18A-C illustrate a suction port configuration of one embodiment of the present invention.

FIGS. 19A-B illustrate tissue being engaged within a suction port configuration shown in FIG. 18-C.

FIG. 20 illustrates a running suture line plicating tissue resulting from a method of practicing one embodiment of the present invention.

### DETAILED DESCRIPTION OF THE INVENTION

The present invention is more particularly described in the following examples with reference to the accompanying drawings that are intended as illustrative only since numerous modifications and variations therein will be apparent to those skilled in the art.

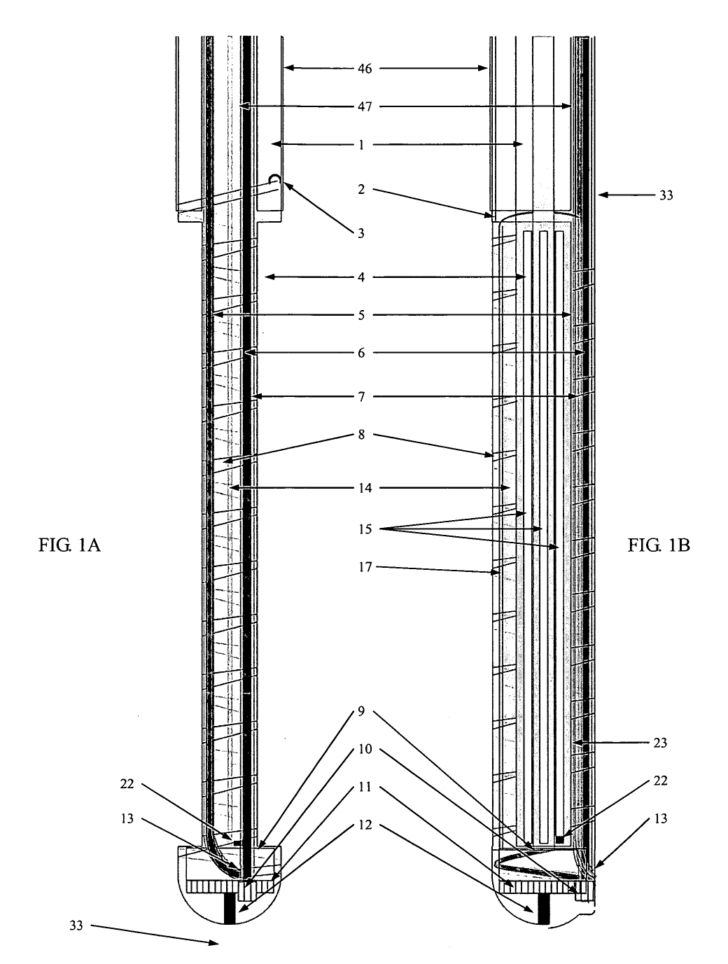

The present invention is a device suitable for placement of sutures, for example, in a suture line. As shown in the exemplary embodiment of FIG. 1, the suturing device of the present invention may include an outer tube 1. The tube 1 has a flexibility to help facilitate insertion, internal navigation and positioning within a patient. The tube 1 can be transparent, translucent, or opaque. The tube 1 has a diameter and flexibility that is amenable for insertion into a natural body orifice, such as the mouth or anus, or into a surgical incision or existing stoma. The diameter can be, for example, about 5 mm to about 22 mm for oral insertion and about 5 mm to about 35 mm for anal insertion.

In this embodiment, the tube 1 has a length that is sufficient to span the distance from its place of insertion to the targeted surgical location. For example, in gastrointestinal (GI) uses, the tube 1 can be approximately two to three feet. This length enables the device to reach several organs within the GI tract or within the abdominal cavity while a proximal end of the tube remains outside the patient's body and accessible by the operator. The tube or enclosure may contain an endoscope, may be attached to an endoscope, may work in concert with an endoscope, or may work independently of an endoscope. The tube or enclosure can be in direct communication with a vacuum source.

In one embodiment, the tube is designed to form two suction ports 4 running vertically on opposing sides of the distal end of the tube. The length of the suction ports can vary from 1 cm to 25 cm. The length of the suction ports can be altered using a sliding sleeve 46 on the exterior of the tube or with “shutters” that can be manipulated to cover a portion of the ports. The diameter of the sleeve's bore is slightly larger than the outside diameter of the main tube. The sleeve may be of a partial circumference, allowing an opening 47 for channels running on the side of the tube. When performing a gastroplasty procedure to reduce the volume of the stomach 30, the suction ports will be approximately 15 cm to 20 cm in length. The width of each suction port can range from 80 to 90 degrees of the tube's circumference. For example, a device having a tube diameter of 18 mm may therefore have suction ports each possessing a width of approximately 12 mm to 14 mm.

The tube may have a helical channel, groove, or track 8 formed within the bore of the tube. The track 8 will start distal to the suction openings 4 and continue up at least as far as the proximal end of the suction openings 4. Typically, the track 8 is integral with the wall of the tube 1 and can be formed directly into the wall. A groove 18 can be formed within the track 8 that which may releasably hold a partition 14, as described in greater detail below.

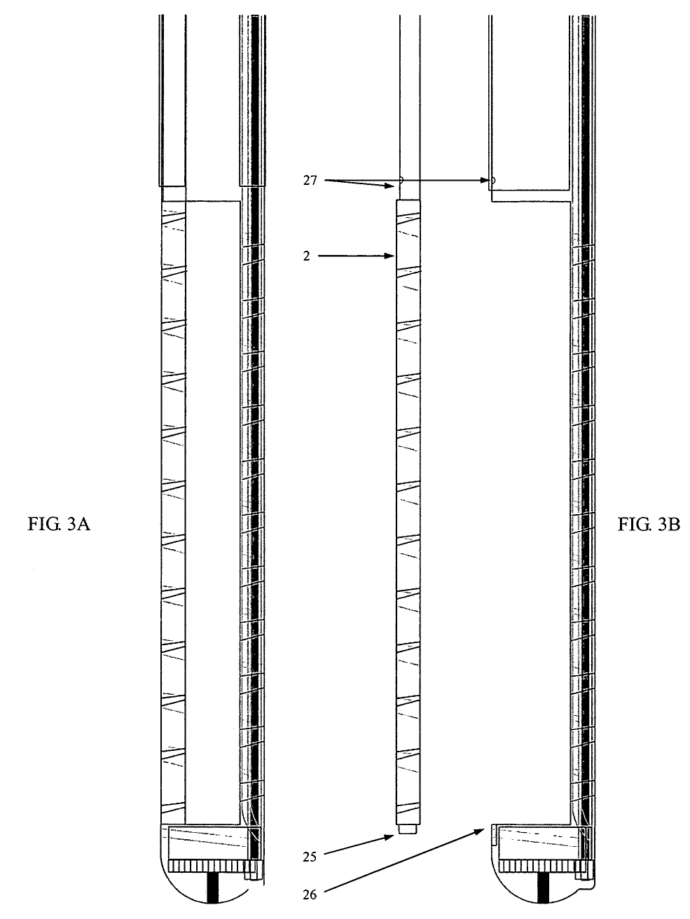

As shown in FIGS. 3A and 3B, the tube 1 is typically made up of two components; a main portion and a detachable portion 2. The main portion can have a closed distal end and generally houses the elements that aid in advancing a needle along the track 8, the needle or needle tip, and balloon catheters. The detachable portion 2 releasably mates with the main portion. The distal end of the detachable portion 2 can have a protrusion 25 that fits into a slot 26 in the distal compartment of the main portion. As such, the detachable portion 2 is temporarily fastened to side one proximal to the suction opening and can be detached when the device is to be dismantled. In one embodiment, eyelets 27 on the two sides line up and can be tied together with a connecting suture (not shown). The connecting suture can be cut to allow the detachable portion 2 enabling side two to be withdrawn up through the main portion of the tube 1. The dismantling of the device allows the device to be withdrawn from the each side of the suture line and prevents the device from effectively being sewn into the tissue and entrapping the device.

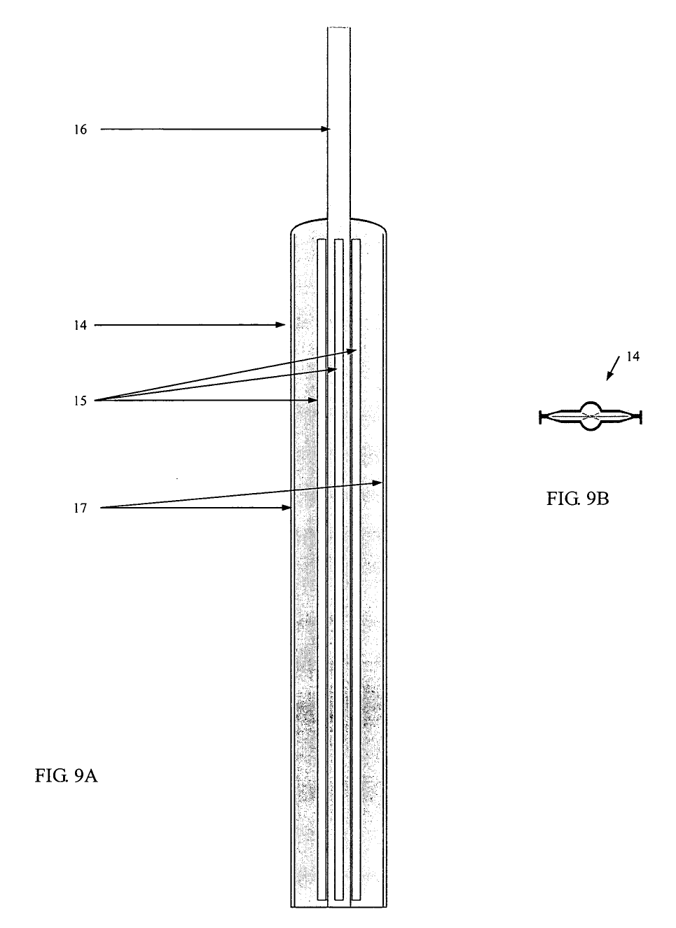

The suction openings 4 are separated by a removable partition 14 that can run down the center of the tube 1 and to effectively create two equal sized cavities on apposing sides of the tube 1. The partition 14 is shown in greater detail in FIGS. 9A and 9B. The partition 14 enables equal amounts of tissue envagination within both suction ports 4. The partition 14 may be hollow or have hollow portions. The partition may house components running within, such as a drive shaft 6 and or balloon catheters 33, 45. The partition may have slots or holes 15 to allow vacuum communication with the tissue. The slots 15 running the length of the partition can insure vacuum communication throughout the entire distance of the suction ports 4.

The partition 14 can also serve to hold the main portion and detachable portion 2 of the tube 1 together and maintain the portions in the proper orientation, particularly in the area of the suction ports 4. The partition can have fins 17, or generally, a shape that is wide at the perimeter and then becomes narrower within the perimeter. The fins 17 on apposing sides of the partition slide vertically into the designed grooves or notches 18 on apposing sides of the tube, similar to an interlocking dovetail joint. The partition 18 can be raised out of the area of the suction ports 14 after a needle has been deployed.

In one embodiment, the partition 14 may be connected to an energy source and have the ability to bum the drawn in tissue at the area of, or along the line of, where the two tissue surfaces will be sewn together, thus enabling the tissues to heal or fuse together.

In one embodiment, the suture needle has a rigid segment 20 and is formed in a spiral or helix shape. The length of the segment 20 is approximately equal to the circumference of the track 8. The rigid segment can be a needle tip 20 with a suture attached. The tip 20 can be releasably attached to a flexible hollow needle shaft 19. For example, the proximal end of the needle tip 20 may have a cavity sized to receive and thereby be releasably held by the needle shaft 19. The suture 21 can run completely through the flexible hollow needle shaft 19. The flexible hollow needle shaft 19 is of a length sufficient to travel at least the length of the track 8 within the tube 1. The flexible hollow needle shaft 19 has a column strength adequate to push the rigid needle tip 20 up the length of the helical track 8 within the tube 1.

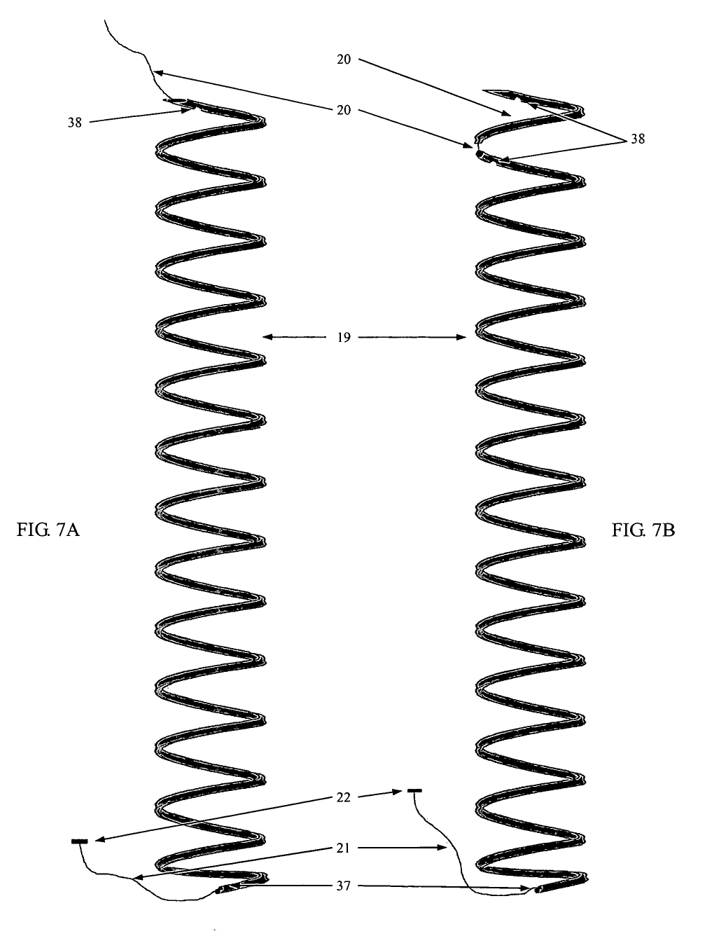

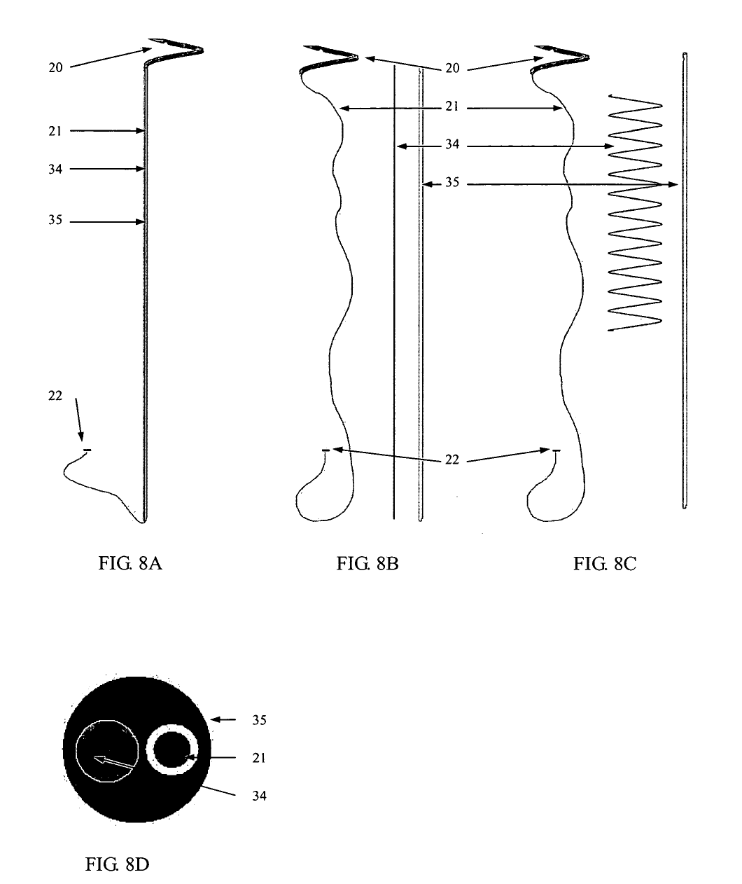

One embodiment of the needle tip 20, needle shaft 19, and suture 21 is shown in FIGS. 7A-B. The flexible hollow needle shaft 19 can be formed out of a metal alloy tubing. Alternatively, the needle shaft 19 can be formed by a composite of materials, such as shown in FIGS. 8A-C. For example, the shaft 19 can include one or more metal or metal alloy wires 34 contained within or incorporated into a tube 35. The tube can be, for example, plastic, silicon, or Teflon. The needle tip 20 creates a helical path within the tube 1 as it is rotated or pushed forward. The body or shaft of the needle will follow the course taken by the tip of the needle. The wire 34 can be formed such that the needle shaft 19 is straight as shown in FIG. 8B, or a coil or helical shape as shown in FIG. 8C. FIG. 8D is a cross-section of the needle shaft 19.

A hollow needle 19 with an internal suture 21 prevents the need to have to pull suture material through multiple bites of tissue. Suture material placed in a spiral fashion within tissue may have a tendency to tear the tissue when the suture is pulled through it.

In another embodiment, the needle shaft and tip are one unit and not hollow. The suture is connected to the proximal end of the needle shaft.

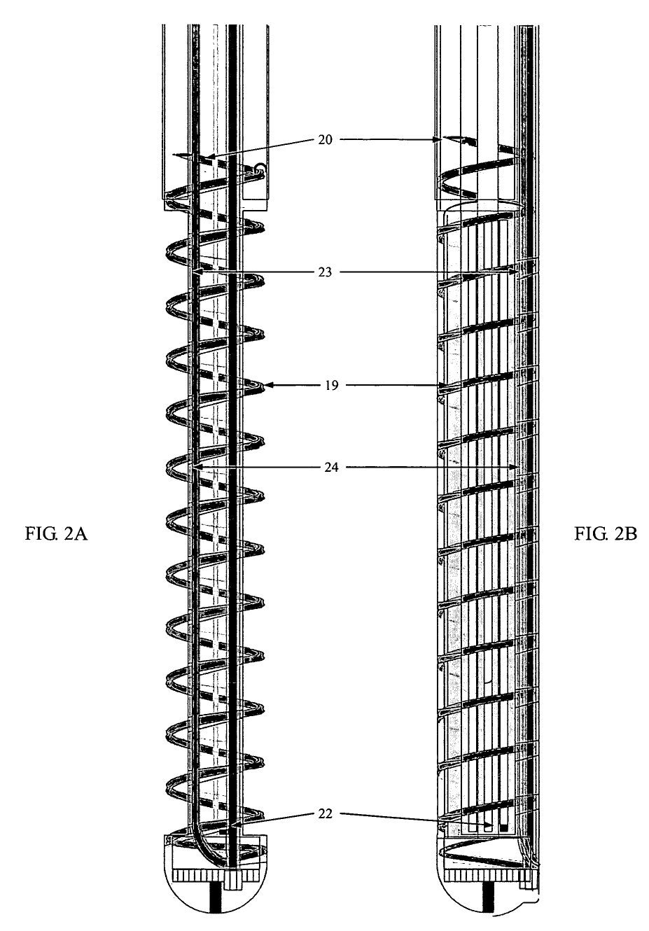

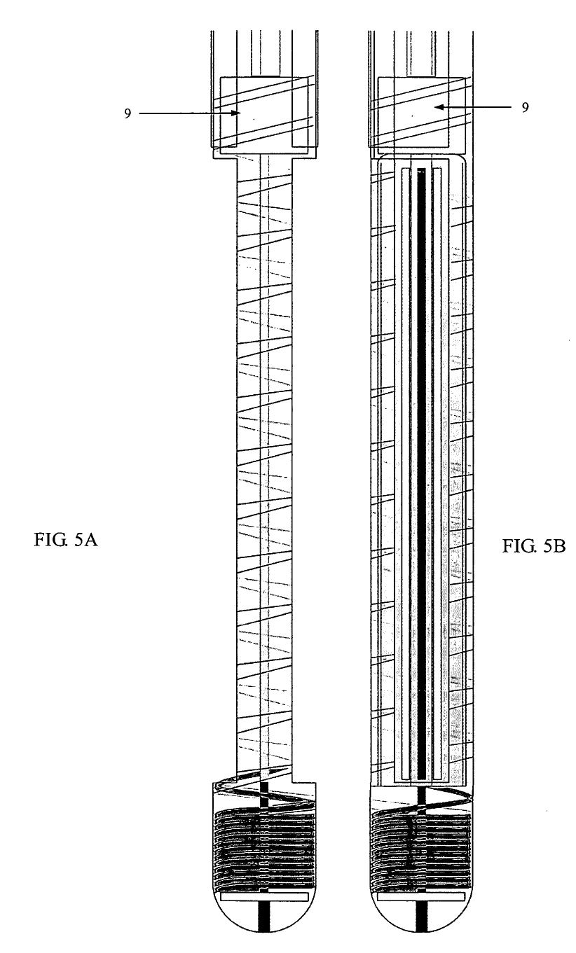

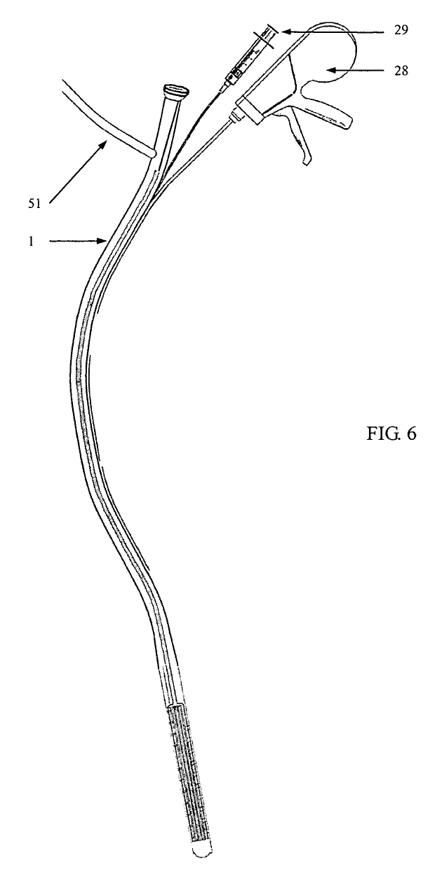

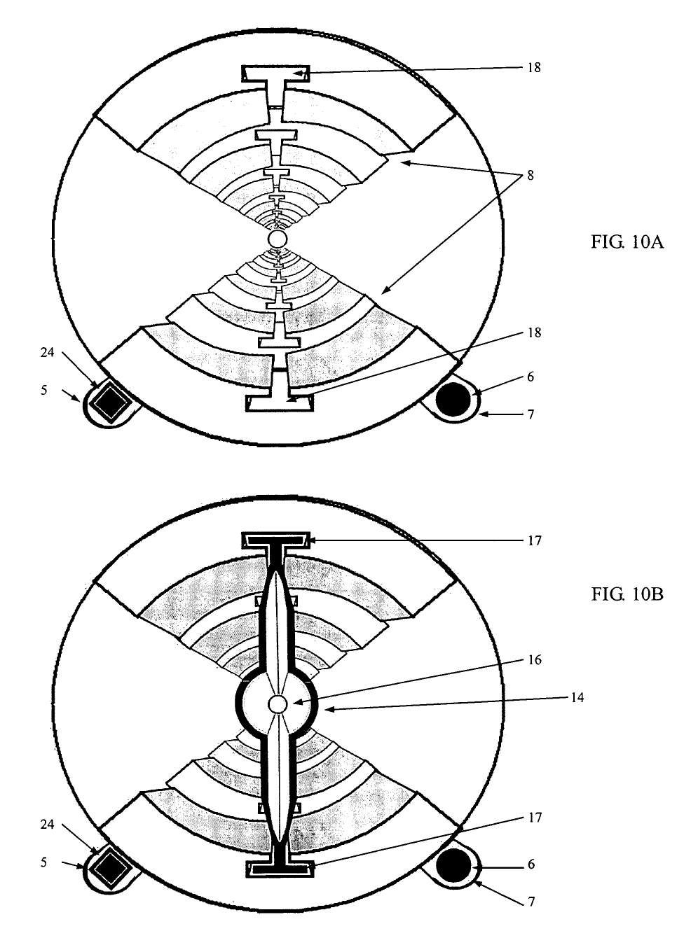

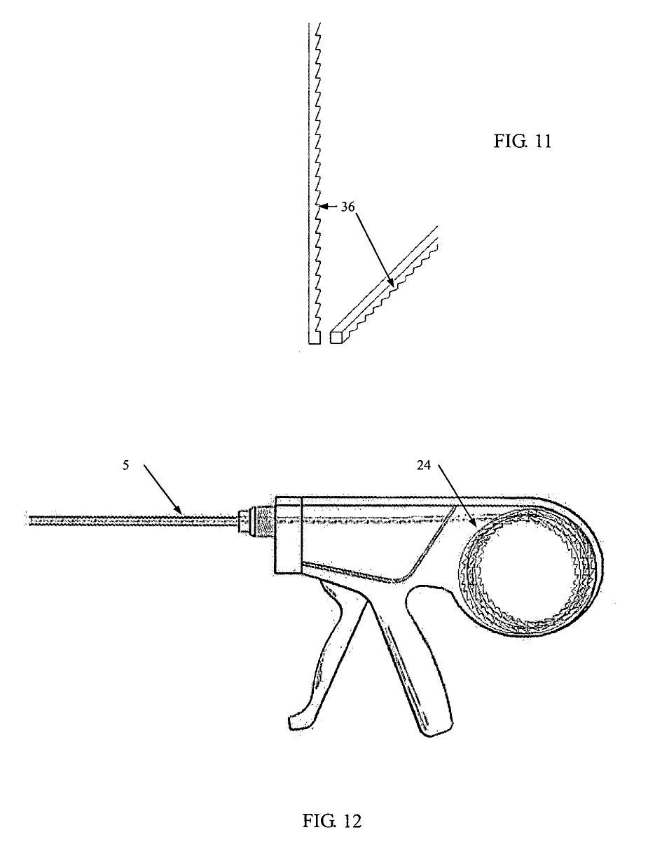

The needle can be propelled or advanced up within the tube's bore in several different ways. In one embodiment shown in FIGS. 1A and 1B, the needle tip 20 is positioned in the distal compartment of the tube 1, beneath the suction ports 4, within the distal end of the helical track 8. The flexible hollow needle shaft 19 runs straight up a cannula 5 which may be located on the side of the tube 1 or alternatively may run up the center of the tube 1 within the removable partition 14. The cannula 5 is a tubular passageway though which material can travel in either a forward or backward direction. The cannula 5 has an internal diameter adequate to contain a pushrod and the flexible suture needle 19 and accompanying suture. The cannula 5 is generally utilized to direct the flexible needle to the distal compartment of the tube 1 where the cannula 5 exits at portion 13 into the internal bore of the tube 1. The cannula 5 also directs the force necessary to advance the needle 19, 20 and contain or prohibit lateral movement. A pushrod 24, which is shown in FIGS. 2A and 2B, may be used to push the needle shaft 19 the length of the cannula 5. The cannula 5 may contain a needle shaft 19 configured as either a straight or coiled shaft. As more clearly shown in FIGS. 11 and 12, the pushrod 24 may have ratchet teeth 36 and be capable of being advanced through the cannula 5 utilizing a ratcheting system such as a ratchet gun 28. FIG. 6 also illustrates the ratchet gun 28 The pushrod may also be advanced manually or using a motor.

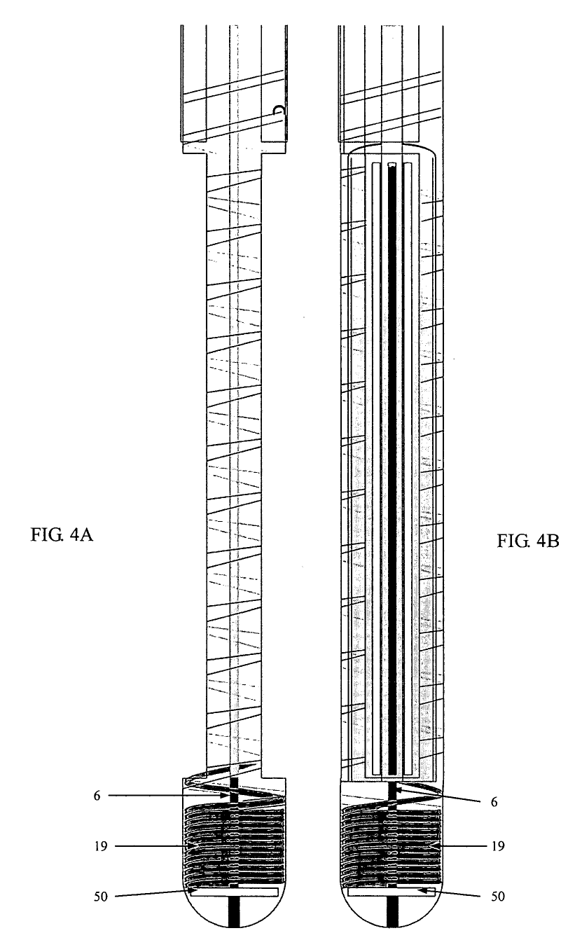

In another embodiment as shown in FIGS. 4A and 4B, the needle shaft 19 is configured as a coil or spiral and is initially contained in the distal compartment of the tube 1. A carriage 50 for detachably holding the coiled needle has the ability to rotate within the distal compartment of the tube. A drive shaft 6 for actuating the carriage may run up the side of the tube (as shown in FIGS. 1A and 1B) or alternatively may run up the center of the tube within the removable partition 14 (as shown in FIG. 4B). The drive shaft 6 can be flexible. If the drive shaft 6 is configured to run up the side of the tube 1, a system of gears 10,11 can be incorporated to actuate the carriage. If the drive shaft 6 is configured to run up the center of the tube 1, it may be releasably attached to the carriage, enabling the drive shaft to be withdrawn up within the tube 1 after a suture line has been placed. Rotating the carriage 50 will propel the spiral needle up the helical track 8 within the bore of the tube 1. The drive shaft may be rotated manually or it may be rotated using a motor.

FIGS. 10A and 10B show the removable partition 14 relative to the drive shaft 7 on the side of the tube 1 and the push rod 5. Although FIGS. 10A and 10B show both the drive shaft 7 and push rod 5, typically these two methods of needle advancement would not be used in the same device.

As shown clearly in FIGS. 7A and 7B, the proximal end of the needle shaft 19 may have a notch 37 to allow the suture material to exit the needle shaft on the side. This can prevent the suture from being severed by pressure applied upon the proximal end of needle shaft by the pushrod or alternatively by the rotating carriage 50.

After the tip 20 of the spiral needle traverses the distance of the suction ports 4, the needle tip 20 can be engaged on the proximal side of the suction ports 4 in order to complete the needle's path. In one embodiment, the detachable needle tip 20 is detached and grasped using an endoscopic grasper or snare (not shown) and pulled upwards within the tube, as the flexible hollow needle shaft 19 is left in place. As the needle tip 20 is withdrawn, it pulls the suture 21 slack through the hollow needle shaft 19, until a suture anchor 22 comes in contact with the tissue at the distal end of the suture line. The needle shaft 19 can now be grasped using a grasper or snare and pulled up within the tube 1. The needle shaft 19 runs through an eyelet or partial eyelet 3 attached to, or formed within the tube 1, which establishes a fulcrum to pull the flexible hollow needle shaft through. The eyelet 3 can be seen, for example, in FIGS. 1A and 2A. The flexible nature of the needle shaft 19 allows the shaft to be pulled up straight on the proximal side of the eyelet 3, while the shaft portion distal to the eyelet 3 continues to travel in a helical path. The hollow nature of the needle shaft 19 allows the suture to remain behind in the tissue extending out the back end of the needle shaft 19 as it is raised within the tube 1.

As shown in FIGS. 7A and 7B, the needle tip and the flexible hollow needle shaft may have a notch or notches 38 to facilitate effective grasping with a cable or wire snare.

The needle, include the needle tip and/or needle shaft, can be manufactured from a material with shape memory.

In another embodiment shown in FIGS. 5A and 5B, the needle is advanced on the proximal side of the suction ports by utilizing a roller 9. The roller 9 engages the needle 19, 20 by pressing the needle tip and shaft between the roller 9 and the interior of the tube 1. The roller 9 is positioned to rotate on a vertical axis within the tube. The roller 9 is of a size complementary to rotating freely within the tube, while applying pressure on the needle tip 20 and shaft 19 when introduced between the roller and internal wall of the tube 1. A drive shaft running centrally within the device tube could rotate the roller 9. The roller 9 may be configured such that a vacuum can pass through it. A roller 9 with a hub and spoke configuration can allow for a vacuum communication between the tube proximal to the roller and the tube distal to the roller. The outer circumference of the roller can be made up of a substance that grips, such as a rubber or elastomer material.

In an alternate embodiment, no mechanism of needle advancement is included. Instead, the needle is a relatively long needle that can be manipulated from outside a patient.

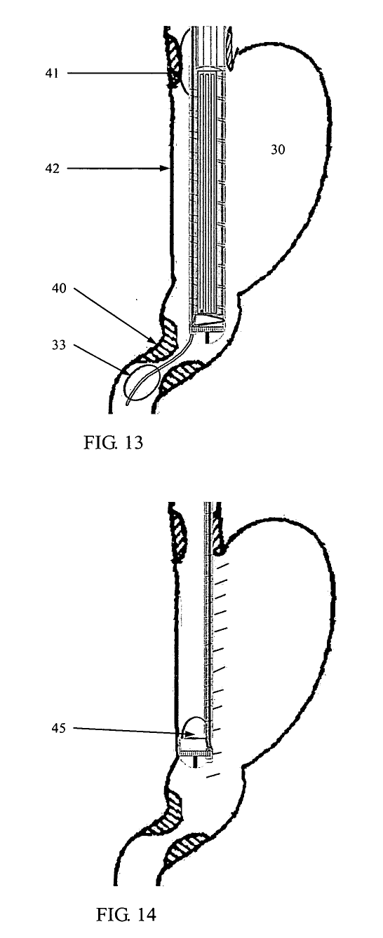

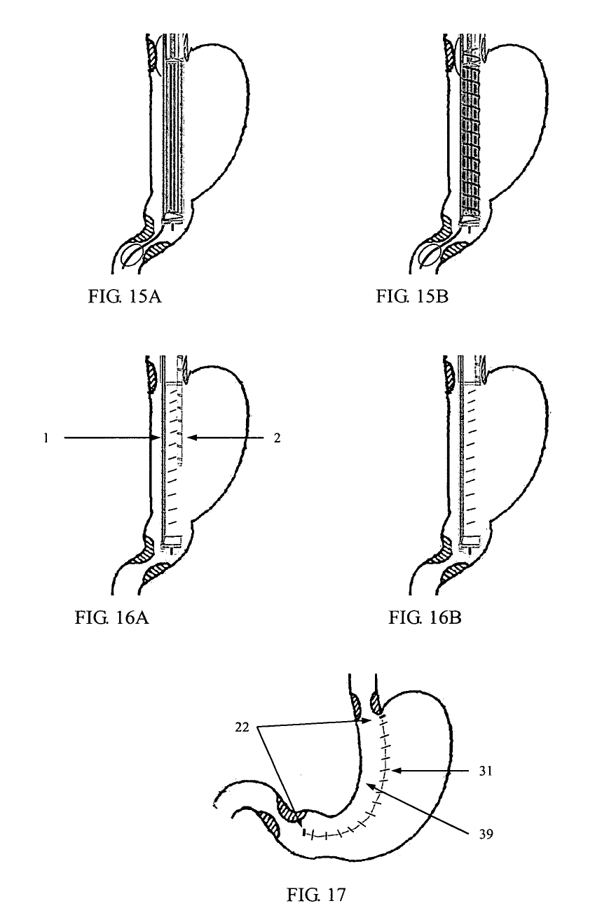

As shown in FIG. 13, the device may utilize a balloon catheter 33 to anchor the device in the desired location within the body organ or cavity. The balloon catheter 33 may run down the side or the center of the tube 1 within a cannula and extend beyond the distal end of the tube. The balloon can be placed in a valve (for example, the pyloric valve 40), outlet or defect and inflated to hold the catheter in place. The device can then slide longitudinally along the catheter until the desired location was reached.

The device can also utilize a balloon catheter 41 to position the device latitudinally. A balloon or inflatable member 41 may be placed on the side of the tube 1 near the proximal end of the suction ports 4. The inflatable member 41 could be positioned above and below the gastric cardia with the balloon directed toward the lesser curvature 42 of the stomach. When inflated, the suction ports 4 could be pushed and positioned properly inline, for example, to create a sleeve gastroplasty.

As shown in FIG. 14, the device may also utilize a balloon catheter 45 to assist in withdrawal of the device. The balloon catheter 45 may be positioned within the distal compartment of the tube 1. When inflated, the balloon extends out of the top of the proximal end of the distal compartment providing a non-angular smooth surface and preventing the distal compartment from catching on tissue as the device is withdrawn.

As shown in FIGS. 15A-B, 16A-B, and 17, in one embodiment in performing a sleeve gastroplasty 39 or stomach volume reduction, the device is inserted into the patient's mouth, advanced through the esophagus and into the stomach. The distal balloon catheter is advanced into the pyloric valve and inflated, anchoring the catheter distally. The tube is advanced, sliding along the shaft of the balloon catheter, until the distal end is the intended distance from the pyloric valve. The balloon catheter located on side one of the device is inflated, thereby pushing the device away from the lesser curvature of the stomach 42 and in line with the location of the intended suture line 43. The vacuum is activated, drawing tissue into the opposing suction cavities. The pushrod is advanced through the cannula thereby pushing the flexible hollow needle shaft held within the cannula. The needle shaft is advanced out the of cannula exit 13 and advances the needle tip up the helical track within the tube. The rigid nature of the needle tip, with its shape complimentary to the helical track, can prevent the needle from deviating from its designed course up the helical track. As the needle spirals up the helical path, it alternately penetrates the tissue held within the opposing suction cavities. When the needle tip emerges on the proximal side of the suction ports, the tip passes through the needle fulcrum and is deflected slightly inward by a protrusion on the interior surface of the tube, facilitating the grasping of the needle tip. The vacuum may be deactivated. A snare may be used for grasping the needle tip. A notch in the needle tip may help facilitate the grasping of the needle tip. The detachable needle tip is pulled up through the tube pulling the attached suture behind it. The slack of the suture is pulled through the flexible hollow needle shaft, which is still incorporated within the tissue. The slack is pulled until the attached suture anchor contacts the tissue at the distal end of the suture line and prevents the suture from being pulled further. The use of a hollow needle shaft prevents the need for pulling suture material through multiple bites of tissue, which may tear the tissue. Tension may be maintained on the suture. The flexible hollow needle shaft may now be grasped. A snare may be used for grasping the needle shaft. A notch in the needle shaft may help facilitate the grasping of the needle shaft. The needle shaft may now be pulled straight upward through the needle eyelet. The flexible nature of the needle shaft allows the portion of the needle shaft distal to the needle eyelet to continue on its helical path as the shaft is withdrawn. The tension maintained on the suture as the needle shaft is withdrawn will remove the slack from the suture line. The tube may now be dismantled into to facilitate its withdrawal from the patient's stomach. The partition is withdrawn up the device tube. The temporary attachment that holds the main and detachable portions together is disengaged. The detachable portion is withdrawn straight up within the main portion of the tube. The distal and side balloons are deflated. Tension is used to eliminate slack in the suture line and a suture anchor can be placed on the suture to secure the proximal end of the suture line. The main portion of the device is rotated so that the open side of the distal tube is facing away from the suture line and the device is withdrawn from the patient's stomach, completing the gastroplasty procedure.

The distal suture anchor 22 may be housed in the distal compartment of the main tube prior to deployment of the suture line. The attached suture 23 may run up the cannula 5 and have a length of string or suture looped through the suture slack with the string or suture running up out of the cannula 5. Tension can be maintained in the suture to eliminate slack.

In the embodiment where the needle shaft is initially housed in the distal compartment of the main tube as a coil, the suture slack may be maintained on a spool within the distal compartment.

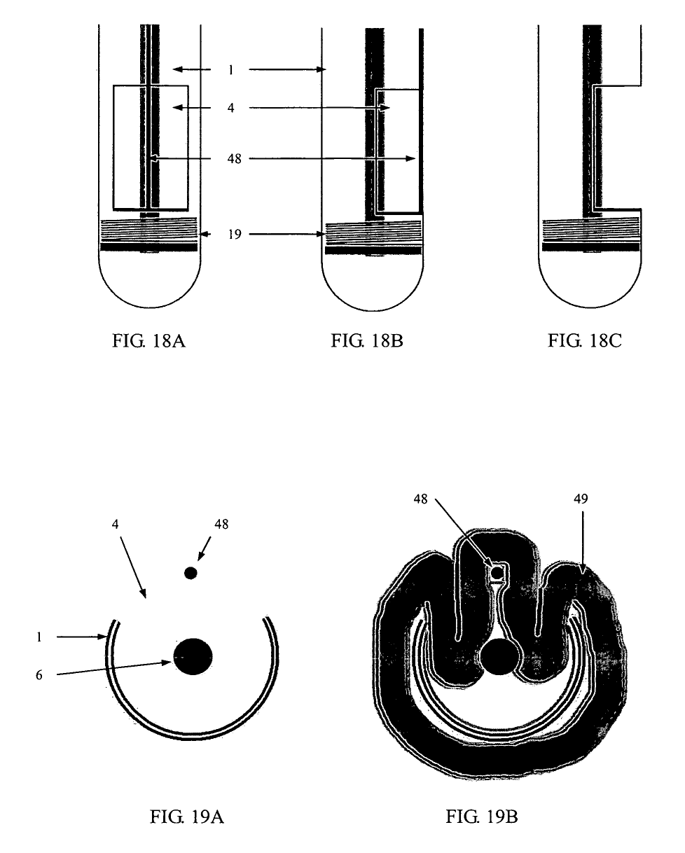

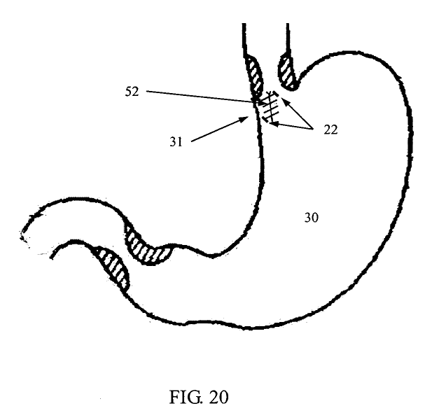

In another embodiment such as shown in FIGS. 18A-C, the device may be configured to place suture in a running suture line to create a plication 52 of tissue as shown in FIG. 20. FIG. 19A is a cross sectional view of a portion of this embodiment. In this embodiment, the tube has one suction port 4 with a removable shaft 48 placed and temporarily held so that it bisects the port 4 vertically, essentially creating two smaller adjacent suction ports. When the vacuum is activated, two envaginations of tissue are drawn into the bore of the tube, as shown in FIG. 19B. The spiral needle is actuated directing the needle tip and attached suture on a helical path through the area of the suction port. The needle penetrates both envaginations of tissue. The needle tip attached to suture is grasped and brought up within the tube as described earlier. The needle shaft is grasped and brought up the tube as described earlier. The removable shaft is lifted or removed from the area of the suction opening, freeing the device from the tissue. The proximal end of the suture line is secured with a suture anchor or the suture is tied to itself within one of the loops of the suture line. This plication may be effective in reducing the diameter of a lumen.

In one embodiment, a portion of the tube of the device may have the ability to decrease and increase its diameter. This could be facilitated by allowing the distal end of the tube to collapse or fold around itself while the two sides of the device could be held side-by-side and then assembled with the removable partition, after the components had been advanced into the stomach. This would allow easy passage of the device through the mouth and down the esophagus, then have the ability to expand to a larger bore once inside the more cavernous organ.

The device can be used in multiple surgical specialties. These specialties may be, but are not limited to, gastrointestinal surgery, cardiac and vascular surgery, gynecological surgery, pulmonary surgery, and general surgery, and may include procedures such as endoluminal gastroesophageal reflux disease procedures such as augmentation of the gastric cardia, gastrointestinal surgery such as gastric reduction or gastroplasty, gastric bypass or gastrojejenoscopy, intestinal anastomosis, gastric excision procedures, outlet reduction, control of gastric bleeding, gastric closure following transgastric surgeries, cardiac valve replacement surgery, mitral valve repair, mitral annulaplasty ring implantation, mitral leaflet “edge-to-edge” valve repair, ventricular remodeling, management of atrial appendage, septal defect repair, graft implantation, vascular anastomosis, fecal incontinence surgery, and hemorrhoid surgery. In an embodiment particularly useful for GI suturing, the device is inserted into the GI tract. In this embodiment, the tube has a diameter that can range, for example, from about 5 mm to about 22 mm for oral insertion or about 5 mm to about 33 mm for anal insertion.

Although the present invention has been described with reference to specific details of certain embodiments thereof, it is not intended that such details should be regarded as limitations upon the scope of the invention except as and to the extend that they are included in the accompanying claims. For example, although a particular feature of the invention is included in the description of one embodiment, that feature is not necessarily a limitation on the scope of the invention. Conversely, a particular feature described in one embodiment can be incorporated into any of the disclosed embodiments.

What is claimed is:

1. A device for suturing tissue of a patient, comprising:
a tube having a distal end, a proximal end, and an exterior surface, the tube defining a bore extending longitudinally within the tube and at least one suction opening in the exterior surface;
a path arranged within an interior of the tube;
a needle arranged at least partially within the bore, wherein the needle is attachable to a suture;
wherein the suction openings are adapted to be connected to a vacuum source such that, when a vacuum is applied, tissue is drawn into the at least one suction opening and at least partially into the bore, and wherein the needle is adapted to advance through the tissue and along the path to place the suture through the tissue.

2. The device of claim 1, wherein the needle is a generally helical shaped needle.

3. The device of claim 1, wherein the path is on the interior surface of the tube and is helical.

4. The device of claim 1, further comprising a rotatable carriage within the tube for advancing the needle.

5. The device of claim 1, further comprising a cannula arranged with the tube.

6. The device of claim 5, further comprising a pushrod for advancing the needle through and out of the cannula and continuing up the path within the tube.

7. The device of claim 1, wherein the device is sized to suture within a vessel.

8. The device of claim 1, wherein the device is sized to suture within an organ.

9. The device of claim 1, wherein the at least one suction opening includes two suction openings, and wherein the device further comprises a removable partition between the suction openings.

10. The device of claim 4, further comprising a drive shaft for rotating the carriage within the tube.

11. The device of claim 1, further comprising the vacuum source.

12. The device of claim 1, wherein the needle is of sufficient length to traverse the length of the drawn in portions.

13. The device of claim 1, wherein the needle is a generally helical shaped needle having coils spaced about 4 mm to about 15 mm.

14. The device of claim 1, wherein the needle is a generally helical shaped needle having coils of about 1 mm to about 6 mm.

15. The device of claim 1, further comprising a removable partition arranged in the bore for vertically separating the suction openings.

16. The device of claim 15, wherein the interior of the tube defines grooves, and wherein the removable partition is adapted to fit into the grooves.

17. The device of claim 15, wherein the removable partition is connectable to an energy source.

18. The device of claim 15, wherein the removable partition is hollow.

19. The device of claim 15, wherein the removable partition is connectable to a vacuum source.

20. The device of claim 1, wherein the suturing device is adapted for forming a running suture line connecting tissue.

21. The device of claim 1, wherein the suturing device is adapted for forming a running suture line plicating tissue.

22. The device of claim 1, wherein the tube is formed by at two portions that can be separated from one another to withdraw the suturing device from the patient.

23. The device of claim 1, further comprising a sleeve arranged on the exterior of the tube for selectively covering the suction openings.

24. The device of claim 1, wherein the sleeve and the needle cooperate in movement.

25. The device of claim 1, wherein the needle is a generally helical shaped, compressible needle.

26. The device of claim 1, wherein the needle is hollow.

27. The device of claim 1, wherein the needle is flexible.

28. The device of claim 1, wherein the needle has the property of shape memory.

29. The device of claim 1, wherein the needle has a detachable tip.

30. The device of claim 1, wherein the needle includes a detachable tip and a needle shaft.

31. The device of claim 30, wherein the needle tip is relatively rigid and the needle shaft is relatively flexible.

32. The device of claim 31, wherein the needle tip is helical.

33. The device of claim 30, wherein the needle shaft is hollow and the needle tip is attachable to a suture that runs through the needle shaft.

34. The device of claim 30, wherein the needle shaft includes a metal wire encased in a flexible non-metal tube.

35. The device of claim 34, wherein the metal wire is generally helical shaped.

36. The device of claim 34, wherein the metal wire is generally straight.

37. The device of claim 26, wherein a helical shaped needle with the property of shape memory can be contained and pushed through a straight cannula.

38. The device of claim 1, wherein the tube is generally straight.

39. The device of claim 1, wherein the tube is generally curved.

40. The device of claim 1, wherein the suture includes an anchor at a distal end.

41. The device of claim 1, wherein the device is adapted to narrow a diameter of at least one portion of an organ and a vessel by plication.

42. The device of claim 1, wherein the tube defines two suction opening on opposite sides of the tube.

43. The device of claim 1, wherein the at least one suction opening is about 4 inches to about 9 inches.

44. The device of claim 1, wherein the at least one suction opening is about 1 inch to about 3 inches.

45. The device of claim 1, further comprising a motor for actuating the needle.

46. The device of claim 4, wherein the carriage is connectable to a motor for actuating the needle.

47. The device of claim 6, wherein the pushrod has ratchet teeth.

48. The device of claim 6, wherein the pushrod is advanced through a cannula using a ratchet system.

49. The device of claim 6, wherein the pushrod pushes a needle shaft contained within a cannula.

50. The device of claim 47, wherein the pushrod is advanced through a cannula using a ratchet gun.

51. The device of claim 50, wherein the ratchet system is motorized.

52. The device of claim 1, further comprising a balloon catheter extending from the distal end of the tube.

53. The device of claim 1, further comprising at least one inflatable balloon to template a working organ size.

54. The device of claim 1, further comprising an inflatable balloon to assist with a removal of the device.

55. The device of claim 1, wherein the suction openings are formed by a single opening divided by a removable strut.

56. The device of claim 55, wherein the removable strut is 1 mm to about 5 mm in width and of sufficient length to traverse the vertical length of the suction opening.

57. The device of claim 1, wherein the tube has a diameter of about 12 mm to about 22 mm.

58. The device of claim 1, wherein the tube has a diameter of about 3 mm to about 32 mm.

59. The device of claim 1, wherein the at least one suction openings includes two suction openings to draw in two portions of tissue, and wherein the needle is adapted to lead a suture through the two portions of tissue.

60. The device of claim 59, wherein the two portions of tissue are part of a body organ such that the suture effectively reduces the volume of the organ.

61. The device of claim 1, wherein the suture results in a narrow sleeve gastroplasty.

62. The device of claim 1, wherein the suture results in an augmented gastro-esophageal junction.

63. The device of claim 1, wherein the tube is generally transparent to enable endoscopic visualization.

64. The device of claim 1, wherein the distal end of the device is closed.

65. The device of claim 1, wherein the needle has a main shaft initially housed in a distal compartment of the tube.

66. The device of claim 1, wherein the needle has a main shaft initially housed in a cannula.

67. The device of claim 1, wherein the needle has a notch allowing suture to rest therein.

68. The device of claim 1, wherein the needle has a notch to facilitate grasping.
